(19) United States
(12) Patent Application Publication
(10) Pub. No.: US 2019/0381220 A1
(43) Pub. Date: Dec. 19, 2019

(54) NEGATIVE PRESSURE SYSTEMS FOR THE MANAGEMENT OF PLEURAL EFFUSION

(71) Applicant: KCI Licensing, Inc., San Antonio, TX (US)

(72) Inventors: Christopher Brian LOCKE, Bournemouth (GB); Michael J. VOSS, Boerne, TX (US); James Killingworth SEDDON, Wimborne (GB); Benjamin Andrew PRATT, Poole (GB)

(21) Appl. No.: 16/474,377

(22) PCT Filed: Jan. 3, 2018
(86) PCT No.: PCT/US2018/012236

§ 371 Date: Jun. 27, 2019

Related U.S. Application Data
(60) Provisional application No. 62/449,415, filed on Jan. 23, 2017.

(51) Int. Cl. A61M 1/00; A61M 1/04

(52) U.S. Cl. CPC A61M 1/0001; A61M 1/04; A61M 2205/3331; A61M 2210/101; A61M 1/0031

(57) ABSTRACT

Systems for the treatment of pleural effusion are disclosed herein. A first system includes a fluid conductor that provides fluid communication with a pleural space of a patient. The first system also includes a canister and a negative-pressure source in fluid communication with the canister. The negative-pressure source pre-charges the canister to a negative-pressure range and maintains the negative-pressure range within the canister while the canister and the fluid conductor are in fluid communication.

### RELATED APPLICATIONS

The present invention claims the benefit, under 35 USC § 119(e), of the filing of U.S. Provisional Patent Application Ser. No. 62/449,415, entitled “Negative-Pressure Systems For The Management Of Pleural Effusion,” filed Jan. 23, 2017. The provisional application is incorporated herein by reference for all purposes.

### TECHNICAL FIELD

The subject matter set forth in the appended claims relates generally to negative-pressure systems and more particularly, but without limitation, to negative-pressure systems, apparatuses, and methods for the management of pleural effusion.

### BACKGROUND

Layers of tissue line the lungs and chest cavity forming the “pleural space” that surrounds the lungs. Although fluid is normally present between these various layers of tissue, that is, within the pleural space, the accumulation of excess fluid can pose serious health risks. The accumulation of such excess fluid is known as a pleural effusion. Notably, a pleural effusion can impair breathing by limiting expansion of the lungs, and leading to other, related complications such as shortness of breath, rapid breathing, and decreased oxygen supply.

Conventionally, treatment or management of the pleural effusion has been by draining at least some of the excess fluid. For example, a drainage fluid conductor inserted into the pleural space of a patient experiencing the pleural effusion may be used to drain the excess fluid. In some instances, such drainage fluid conductors have been connected to a canister charged to a particular negative pressure, such as a radon bottle. However, such conventional canisters suffer from numerous shortcomings, and improvements to systems, components, and processes for treating or managing pleural effusion may benefit healthcare providers and patients.

### BRIEF SUMMARY

New and useful systems, apparatuses, and methods for the treatment of a pleural effusion are set forth in the appended claims. Illustrative embodiments are also provided to enable a person skilled in the art to make and use the claimed subject matter.

For example, some embodiments disclosed herein relate to a first system for the treatment of pleural effusion. The first system may comprise a fluid conductor configured to provide fluid communication with a pleural space of a patient. The first system may further comprise a canister and a negative-pressure source in fluid communication with the canister. The negative-pressure source may be configured to pre-charge the canister to a negative-pressure range and to maintain the negative-pressure range within the canister while the canister and the fluid conductor are in fluid communication.

Additional or alternative embodiments also disclosed herein relate to a second system for the treatment of pleural effusion. The second system may comprise a fluid conductor configured to provide fluid communication with a pleural space of a patient. The second system may further comprise a pump head and a power unit detachably coupled to the pump head so as to impart rotational power to the pump head when coupled. The second system may still further comprise a collection vessel. The pump head may be positioned along a route of fluid communication between the fluid conductor and the collection vessel.

Objectives, advantages, and a preferred mode of making and using the claimed subject matter may be understood best by reference to the accompanying drawings in conjunction with the following detailed description of the illustrative embodiments.

### BRIEF DESCRIPTION OF THE DRAWINGS

FIG. 1 is a functional diagram of a system according to a first embodiment disclosed herein;

FIG. 2 is a representation of an example embodiment of the pleural effusion treatment apparatus of FIG. 1;

FIG. 3 is a block flow diagram of a process according to one or more embodiments disclosed herein;

FIG. 4 is a block flow diagram of a pleural effusion treatment method according to a first embodiment disclosed herein;

FIG. 5 is a representation of a system according to a second example embodiment disclosed herein;

FIG. 6 is a representation of an alternative embodiment of collection vessel as may be used in a system like the system of FIG. 5;

FIG. 7 is a representation of another alternative embodiment of collection vessel as may be used in a system like the system of FIG. 5;

FIG. 8 is a representation of a pump unit as may be used in a system like the system of FIG. 5 according to a first embodiment disclosed herein;

FIG. 9A is a first view of a representation of a pump unit as may be used in a system like the system of FIG. 5 according to a first embodiment disclosed herein;

FIG. 9B is a second view of a representation of the pump unit of FIG. 9A; and

FIG. 10 is a block flow diagram of a pleural effusion treatment method according to a second embodiment disclosed herein.

### DESCRIPTION OF EXAMPLE EMBODIMENTS

The following description of various example embodiments provides information that enables a person skilled in the art to make and use the subject matter set forth in the appended claims, but may omit certain details already well-known in the art. The following detailed description is, therefore, to be taken as illustrative and not limiting.

The example embodiments may also be described herein with reference to spatial relationships between various elements or to the spatial orientation of various elements depicted in the attached drawings. In general, such relationships or orientation assume a frame of reference consistent with or relative to a patient in a position to receive treatment. However, as should be recognized by those skilled in the art, this frame of reference is merely a descriptive expedient rather than a strict prescription.

The term “about,” as used herein, is intended to refer to deviations in a numerical quantity that may result from various circumstances, for example, through measuring or handling procedures in the real world; through inadvertent error in such procedures; through differences in the manufacture, source, or purity of compositions or reagents; from computational or rounding procedures; and the like. Typically, the term “about” refers to deviations that are greater or lesser than a stated value or range of values by 1/10 of the stated value(s), e.g., ±10%. For instance, a concentration value of “about 30%” refers to a concentration between 27% and 33%. Each value or range of values preceded by the term “about” is also intended to encompass the embodiment of the stated absolute value or range of values. Whether or not modified by the term “about,” quantitative values recited in the claims include equivalents to the recited values, for example, deviations from the numerical quantity, but would be recognized as equivalent by a person skilled in the art.

In general, fluids which may include exudates from a tissue site flow from relatively higher pressure toward relatively lower pressure along a fluid path. Thus, the term “downstream” typically implies something in a fluid path relatively closer to a source of negative pressure or further away from a source of positive pressure. Conversely, the term “upstream” implies something relatively further away from a source of negative pressure or closer to a source of positive pressure. Similarly, in some instances, certain features may be described in terms of a fluid “inlet” or “outlet” in such a frame of reference. This orientation is generally presumed for purposes of describing various features and components herein. However, the fluid path may also be reversed in some applications (such as by substituting a positive-pressure source for a negative-pressure source) and this descriptive convention should not be construed as a limiting convention.

### Pleural Effusion Systems—First System

Disclosed herein are one or more embodiments of systems useful in the treatment of a pleural effusion, for example, referred to as a system. The term “treatment,” as used herein, is intended to be broadly construed, for example, to any include any measure or series of measures intended to manage, treat, alleviate, delay the onset of, suppress, or as a prophylactic with respect to the disease states, disorders, or conditions discussed herein, as well as any sign or symptom associated therewith or resulting directly or indirectly therefrom.

In some embodiments, a system may comprise a fluid conductor, such as a tube, configured to provide fluid communication with a pleural space of a patient, a canister, and a negative-pressure source configured to precharge the canister to a negative pressure and to maintain the negative pressure within the canister while the canister and the fluid conductor are in fluid communication.

The term “negative pressure” as used herein is intended to generally refer to a pressure less than a local ambient pressure, such as the ambient pressure in a local environment of a treatment system or apparatus. In many cases, the local ambient pressure may also be the atmospheric pressure at which a tissue site is located. Alternatively, the pressure may be less than a hydrostatic pressure associated with tissue at the tissue site, for example, the hydrostatic pressure within the pleural space of a patient. Unless otherwise indicated, values of pressure stated herein are gauge pressures. Similarly, references to increases in negative pressure typically refer to a decrease in absolute pressure, while decreases in negative pressure typically refer to an increase in absolute pressure. While the amount and nature of negative pressure may vary according to therapeutic requirements, the pressure is generally a low vacuum, also commonly referred to as a rough vacuum, between −5 mm Hg (−667 Pa) and −500 mm Hg (−66.7 kPa).

The fluid mechanics associated with the use of a negative-pressure source to reduce pressure in another component or location can be mathematically complex. However, the basic principles of fluid mechanics applicable to negative-pressure therapy are generally well-known to those skilled in the art, and the process of reducing pressure may be described illustratively herein as “delivering,” “distributing,” or “generating” negative pressure, for example.

Two or more components of the apparatuses and/or systems disclosed herein may be fluidly coupled to each other to provide a path for transferring fluids, such as a liquid and/or gas, between those components. For example, components may be fluidly coupled through a fluid conductor. A “fluid conductor,” as used herein, broadly includes a tube, pipe, hose, conduit, or other structure with one or more lumina adapted to convey a fluid between two ends thereof. Typically, a fluid conductor is an elongated, cylindrical structure with some flexibility, but the geometry and rigidity may vary. In some embodiments, components may also be coupled by virtue of physical proximity, for example, being integral to a single structure, or being formed from the same piece of material. Moreover, some fluid conductors may be molded into or otherwise integrally combined with other components. Additionally, a fluid conductor, such as a tube, between two or more components may also include mechanical, thermal, electrical, or chemical coupling (such as a chemical bond) in some contexts. For example, a fluid conductor may mechanically and fluidly couple two components, in some embodiments.

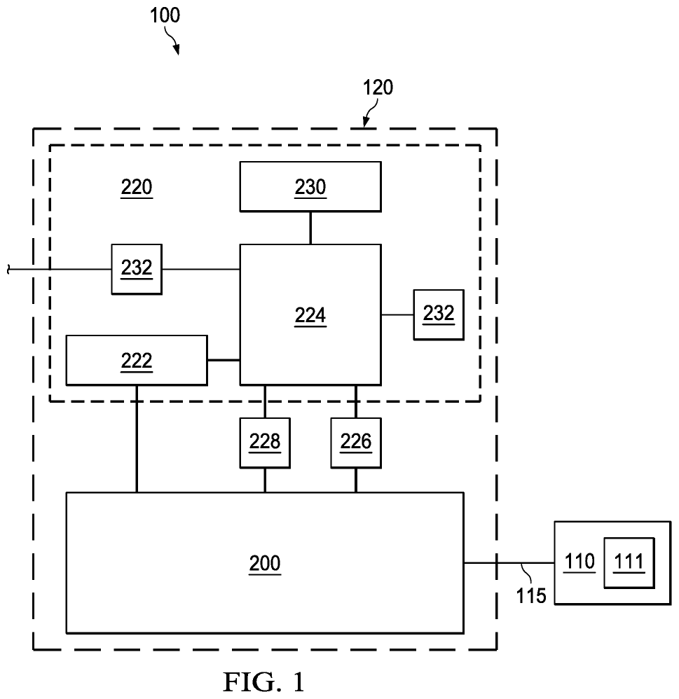

For example, FIG. 1 schematically illustrates a first embodiment of a system 100 that can provide treatment or management of a pleural effusion. In the embodiment of FIG. 1, the system 100 generally includes a drainage fluid conductor 110 and a pleural effusion treatment apparatus 120. Also in the embodiment of FIG. 1, the system 100 may include an intermediate fluid conductor 115 adapted to provide fluid communication between the drainage fluid conductor 110 and the pleural effusion treatment apparatus 120. The drainage fluid conductor 110 may be generally configured to provide fluid communication with the pleural space of a patient. While in the embodiment of FIG. 1 the intermediate fluid conductor 115 is fluidly in-line between the drainage fluid conductor 110 and the pleural effusion treatment apparatus 120, in other embodiments the intermediate fluid conductor 115 may be omitted, for example, depending upon the proximity between the pleural effusion treatment apparatus 120 and the drainage fluid conductor 110, if the pleural effusion treatment apparatus 120 and the drainage fluid conductor 110 are sufficiently close. In still other embodiments, multiple intermediate fluid conductors 115 may be similarly employed, again depending upon the proximity between the pleural effusion treatment apparatus 120 and the drainage fluid conductor 110.

### System 1—Pleural Effusion Treatment Apparatus

In some embodiments, the pleural effusion treatment apparatus 120 may generally include fluid container, such as a canister, and a therapy unit in fluid communication with the container. The therapy unit may be generally adapted to charge the container to a specific negative pressure, for example, to a target negative pressure within a negative-pressure range, and to maintain the container within the negative-pressure range. For example, if the container is connected to the drainage fluid conductor 110 and fluid communication is allowed, fluid may be drawn into the container, for example, which may be pre-charged to a negative pressure via the negative pressure in the container. The therapy unit may control the pressure within the container so as to maintain the container within the negative-pressure range.

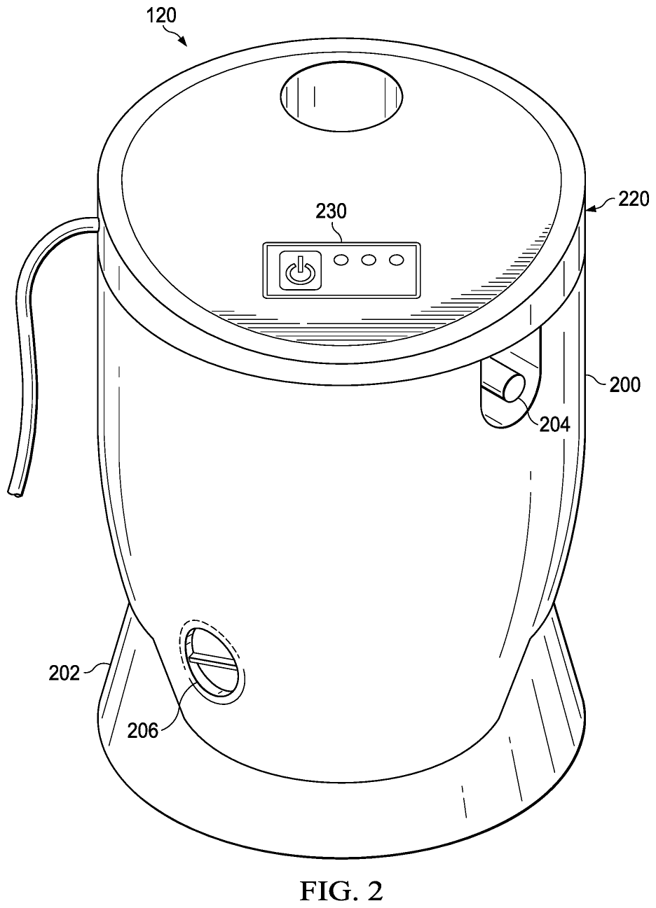

In the embodiment of FIG. 1, the pleural effusion treatment apparatus 120 includes a canister 200 and a therapy unit 220. In the embodiment of FIG. 1, the canister 200 and the therapy unit 220 are integrated together, such as in a single assemblage, to form the pleural effusion treatment apparatus 120. For example, FIG. 2 illustrates additional details that may be associated with some embodiments of the pleural effusion treatment apparatus 120 of FIG. 1. In the embodiment of FIGS. 1 and 2, the canister 200 and the therapy unit 220 may be removably coupled together, for example, such that either the canister 200 or the therapy unit 220 may be removed as needed, for example, so as to be serviced, emptied, cleaned, replaced, or the like. The canister 200 and the therapy unit 220 may be coupled via any suitable engagement, examples of which include, but are not limited to, a threaded connection, a twist-locking connection, and a connection comprising one or more latches, such as draw latches.

In alternative embodiments, any container and source of negative pressure coupled such that the therapy unit may charge the container to a specific negative pressure may be similarly employed. For example, in such alternative embodiments, an operatively-coupled canister and source of negative pressure may be coupled via a route of fluid communication and, in some embodiments, via a route of signal communication. In such alternative embodiments, the canister and source of negative pressure need not constitute a single unit of assemblage, as disclosed with respect to FIG. 2, but may be non-integral components.

### Canister

The canister 200 is an example embodiment of a fluid container, which may be generally configured to define an internal volume, for example, a suitable or desirable internal volume. In some embodiments, for example, the internal volume may be from about 0.5 L to about 2.5 L or, in a more particular embodiment, from about 1.0 to about 1.5 L. The internal volume may be generally adapted to be substantially fluid-tight, for example, such that a negative pressure applied to the canister 200, for example, via operation of the therapy unit 220, may be retained with little dissipation of the negative pressure. For example, the canister may be fluid-tight such that an application of negative pressure to the canister may be maintained such that the negative pressure does not deviate by more than 10% from that pressure for at least about 8 hours. As shown in the embodiment of FIG. 2, the canister 200 and the therapy unit 220 may be coupled together such that the canister 200 and the therapy unit 220 cooperatively define the internal volume. For example, the therapy unit 220 may be sealingly engaged with the therapy unit 220 so as to form a cover or lid to the canister 200. The engagement between the therapy unit 220 and the canister 200 may include a suitable seal, examples of which include but are not limited to, an O-ring, a T-seal, a gasket, and a compression seal, as suitable depending upon the engagement between the therapy unit 220 and the canister 200.

The canister 200 may have any suitable shape, design, and orientation. In some embodiments, for example, the canister 200 may be described as generally conical, tapered, pyramidal, or generally cubic. Also, the canister 200 may be described as having a cross-section in a horizontal plane that is circular, ovular, square, rectangular, triangular, pentagonal, hexagonal, or the like. In some embodiments, the canister may include one or more additional, suitable structural features. For example, in the embodiment of FIG. 2, the canister 200 includes a base 202, for example, to improve the upright stability of the canister 200.

The canister 200 may also be generally configured to provide a suitable route of fluid communication to or from another component. In some embodiments, the canister 200 may further include various fluid ports. For example, in the embodiment of FIG. 2, the canister 200 includes a connection port 204, generally configured to allow fluid communication with the drainage fluid conductor 110, for example, either directly or via the intermediate fluid conductor 115. The connection port 204 may be adapted to be coupled to the drainage fluid conductor 110 or the intermediate fluid conductor 115. In various embodiments, the connection port 204 may include a suitable fitting or coupler. Examples of such fitting and couplers may include, but are not limited to, push-to-connect fittings, compression fittings, barb fittings, or the like.

In some embodiments, the connection port 204 may be adapted so as to disallow fluid communication in at least one direction, such as fluid flow into the canister 200, when a tubing is not coupled with the connection port 204 and to allow fluid communication when a tubing is coupled with the connection port 204. In such embodiments, fluid flow into the canister 200 via the connection port 204, for example, fluid flow toward to the negative-pressure, source, may be precluded via a valve when a tubing such as the drainage fluid conductor 110 or the intermediate fluid conductor 115 is not connected to the connection port 204. For example, in some embodiments the connection port 204 may be configured as a “duck-billed valve, a float valve, or a flap valve. Additionally or alternatively, in some embodiments, the connection port 204 may be configured to provide a signal indicating connection to tubing, such as the drainage fluid conductor 110 or intermediate fluid conductor 115. In such embodiments, the connection port 204 may include, for example, contacts generally configured to provide a signal upon the completion of an electrical pathway by the contacts when the connectors are joined, or other sensors such as pressure switches.

Also, in some embodiments, the intermediate fluid conductor 115 may be configured to be coupled to the drainage fluid conductor 110 via a suitable fitting or coupler which may likewise be adapted so as to disallow fluid communication in at least one direction when the intermediate fluid conductor 115 is not coupled with another tubing, such as the drainage fluid conductor 110, and to allow fluid communication when the intermediate fluid conductor is coupled to the other tubing. Additionally or alternatively, the intermediate fluid conductor 115 may also be configured to output a signal indicative of the intermediate fluid conductor being connected to another tubing.

Also, in some embodiments, the canister 200 may be configured to provide one or more additional routes of fluid communication. For example, in the embodiment of FIG. 2, the canister 200 includes a drain port 206. The drain port 206 is located toward the bottom of the canister 200 and is adapted to allow a fluid contained within the internal volume of the canister 200 to be drained therefrom. While in the embodiment of FIG. 2 the drain port 206 is illustrated as a removable plug, in alternative suitable embodiments, a drain port may include a valve, such as a gate valve or petcock valve, that may be opened to allow fluid to be drained from the internal volume of the canister 200.

### Therapy Unit/Negative-Pressure Source

The therapy unit 220 may be generally configured to charge the canister 200 to a specific negative pressure, for example, a target negative pressure or a negative-pressure range, and to maintain the negative pressure at the target negative pressure or within the negative-pressure range, for example, during operation. In some embodiments, the negative-pressure range may be from about −30 mmHg to about −120 mmHg. In some more particular embodiments the negative-pressure range may be from about −50 mmHg to about −100 mmHg. Referring to again to FIG. 1, the therapy unit 220 generally includes a negative-pressure source 222 and a controller 224.

In some embodiments, the negative-pressure source 222 may be a reservoir of air at a reduced pressure, or may be a manual or electrically-powered device that, when operated, can reduce the pressure within a sealed volume. Suitable examples of the negative-pressure source 222 may include, but are not limited to, a vacuum pump, a suction pump, a wall-suction port available at many healthcare facilities, or a micro-pump. While the amount and nature of negative pressure applied to the canister may vary according to therapeutic requirements, the negative-pressure source 222 may be characterized as generally capable of achieving negative pressures within the range from about −30 mmHg to about −120 mmHg. A narrow or more specific negative pressure to which the canister 200 may be charged may be a reduced pressure at which a caregiver has determined optimal therapy of the pleural effusion may be provided, for example, a therapy pressure. In some embodiments, the negative-pressure source 222 may be configured to use a therapy pressure as a target pressure, for example, such that the negative-pressure source is configured to charge the canister 200 to about the target pressure, more particularly, negative pressure, and to maintain the canister 200 at about the target pressure. For example, the negative-pressure source 222 may be configured to operate so as to reduce the pressure in canister 200 to about the target pressure and, if the pressure in the canister deviates from the target pressure, the negative-pressure source 222 may operate to lower the pressure back to about the target pressure.

In some embodiments, the controller 224 may be communicatively coupled to various other components of the therapy unit 220, for example, so as to control operation of one or more of these components. For example, in the embodiment of FIG. 1, the therapy unit 220 further includes a pressure sensor 226, an engagement sensor 228, and a user interface 230. As shown, each of the negative-pressure source 222, the pressure sensor 226, the engagement sensor 228, and the user interface 230 are in signal communication with the controller 224. The term “signal communication,” as used herein, may refer to a coupling between components that permits the transmission of signals between those components. In various embodiments, the signals may be discrete signals or continuous signals. The signals may also be analog signals or digital signals.

In some embodiments, the signal communication between the controller 224 and another component or device may be one-way communication, for example, such that signals may only be sent in one direction. For example, a sensor may generate a signal that may be communicated to the controller 224, but the controller 224 may not be capable of sending a signal to the sensor. Alternatively, in some embodiments, the signal communication between the controller 224 and another device or component may be two-way communication, for example, such that signals may be sent in both directions. For example, the controller 224 and a user interface may be communicatively coupled so that the controller 224 may send and receive signals from the user interface and, likewise, a user interface may send and receive signals from a controller 224.

In some embodiments, the pressure sensor 226 may be generally configured to detect a pressure and to output a signal indicative of that pressure. For example, in the embodiment of FIG. 1, the pressure sensor is in fluid communication with the canister 200, for example, such that the pressure sensor 226 is configured to detect the pressure within the canister 200, and to output a signal indicative of the pressure within the canister 200. The pressure sensor 226 may include any suitable type or configuration of sensor. For example, in various embodiments, the pressure sensor 226 may be a piezoresistive strain gauge, a capacitive sensor, an electromagnetic sensor, a piezoelectric sensor, an optical sensor, or a potentiometric sensor. In some embodiments, the pressure sensor 226 may measure a strain caused by an applied pressure. For example, such a pressure sensor may be calibrated by relating a known amount of strain to a known pressure applied and, the relationship may be used to determine an unknown applied pressure based on a measured amount of strain. In some embodiments, the pressure sensor 226 may include a receptacle configured to receive an applied pressure.

In some embodiments, the engagement sensor 228 may be generally configured to detect engagement between the therapy unit 220 and the canister 200 and to output a signal indicative of that engagement. For example, the engagement sensor 228 may output a signal indicating that the therapy unit 220 is engaged with the canister 200, a signal indicating that the therapy unit 220 is not engaged with the canister 200, or both. In various embodiments, the engagement sensor 228 may include any suitable type or configuration of sensor. For example, the engagement sensor 228 may be a contact sensor. For example, in embodiment of FIG. 1 the engagement sensor 228 is included such that, as will be disclosed herein, the controller 224 may control the operation of the negative-pressure source 222 dependent upon whether or not the therapy unit 220 is engaged with the canister 200. In some embodiments, the engagement sensor 228 may be configured as a switch. In such an embodiment the engagement sensor 228 may be configured such that the negative-pressure source 222 is operable when the therapy unit 220 is engaged with the canister 200 and is not operable when the therapy unit 220 is not engaged with the canister 200. For example, in such an embodiment, the engagement sensor 228 may be configured such that the therapy unit 220 or a component thereof receives power when the therapy unit 220 is engaged with the canister 200 and does not receive power when the therapy unit 220 is not engaged with the canister 200.

The user interface 230 may be generally configured to allow communication between the controller 224 and an environment external to the system 100. In some embodiments, such an external environment may include an operator or, additionally or alternatively, a computer system configured to interface with the system 100, for example. In some embodiments, the user interface 230 may be configured to receive a signal from the controller 224 and to present information derived from the signal in a manner that may be received by the external environment, such as by a user within the external environment, for example, a physician, care-giver, or patient. Additionally, in some embodiments, the user interface 230 may be configured to receive input from the external environment and, in response, send signals indicative of the input to the controller 224.

In some embodiments, the user interface 230 may include a graphical user interface, a touchscreen, and/or one or more motion tracking devices. For instance, the user interface 230 may also include one or more display screens, such as a liquid crystal display (“LCD”), lighting devices, such as light emitting diodes (“LED”) of various colors, and audible indicators, such as a whistle, configured to emit a sound that may be heard by an operator. Additionally, in some embodiments, the user interface 230 may further include one or more devices, such as knobs, buttons, keyboards, remotes, such as a remote control in signal communication with a suitable transceiver, touchscreens, ports that may be configured to receive a discrete or continuous signal from another device, or other devices configured to permit the user interface to interact with 230 the external environment. For example, the user interface 230 may permit the external environment, for example, a user within the external environment, such as a physician, care-giver, or patient, to select a therapy regimen, for example, having a particular characteristic, such as target pressure, or duration, to be performed via the pleural effusion treatment apparatus 120. In some embodiments, the user interface 230 may display information to the external environment such as a therapy duration, a type of therapy, an amount of reduced pressure being supplied, a fluid level of the canister, or available battery-life, for example.

Also, in the embodiment of FIG. 1, the therapy unit 220 includes a power source 232. The power source 232 may be a device that supplies electrical power to an electric load, such as the controller 224 or the negative-pressure source 222. The power source 232 may supply electrical power to any suitable components, for example, a controller, a sensor, a flow meter, a valve, a user interface, or a pump. In various embodiments, the power source may include a battery, a direct current (DC) power supply, an alternating current (AC) power supply, such as a wall-plug, a linear regulated power supply, a switched-mode power supply, or combinations thereof. For example, in some embodiments, a therapy unit may include both an AC power supply and a battery that is charged when AC power is available and supplies power to one or more components of the therapy unit when AC power is unavailable.

Additionally, in some embodiments, the therapy unit 220 may further include one or more moisture sensors. The moisture sensor, which may also be referred to as a hygrometer, may be a device configured to measure a relative humidity of a space. In some embodiments, a moisture sensor may be a capacitive humidity sensor configured to measure the effect of humidity via a dielectric constant of a polymer or metal oxide material. In other embodiments, a moisture sensor may be a resistive humidity sensor that is configured to measure an electrical resistance of a material as it changes in response to humidity.

Additionally, in some embodiments, the therapy unit 220 may further include one or more valves. In such embodiments, a valve may be generally configured to selectively permit fluid flow therethrough. In various embodiments, such a valve may be a ball valve, a gate valve, a butterfly valve, or other valve type that may be operated to control fluid flow therethrough. Generally, a valve may include a valve body having a flow passage, a valve member disposed in the flow passage and operable to selectively block the flow passage, and an actuator configured to operate the valve member. For example, the actuator may be configured to position the valve member in a closed position, preventing fluid flow through the flow passage of the valve; an open position, permitting fluid flow through the fluid passage of the valve; or a metering position, permitting fluid flow through the flow passage of the valve at a selected flow rate. In some embodiments, the actuator may be a mechanical actuator configured to be operated by an operator. In some embodiments, the actuator may be an electromechanical actuator configured to be operated in response to the receipt of a signal input. For example, the actuator may include an electrical motor configured to operate upon receipt of a signal from a controller. In response to the signal, the electrical motor of the actuator may move the valve member of the valve. For example, in some embodiments, the connection port 204 in the canister may include a valve configured to control fluid communication via the connection port 204. Additionally or alternatively, in some embodiments, fluid communication with the internal space of the canister 200, fluid communication via a particular route of fluid communication may be controlled by a valve included within the therapy unit 220, for example, such that the controller 224 may be configured to operate the valve so as to control fluid communication with the internal space of the canister 200. Additionally or alternatively, in some embodiments, the negative-pressure source 222 source may include one or more valves, for example, configured to permit venting of the canister 200 by allowing ambient air to flow through the negative-pressure source 222 to the canister 200.

Additionally, in some embodiments, a therapy unit 220 may further include one or more flow meters. A flow meter may be any device configured to determine a fluid flow rate. In various embodiments, the flow meter may include a mechanical flow meter, a differential pressure based flow meter, an optical flow meter, an open channel flow meter, a thermal mass flow meter, a vortex flow meter, electromagnetic, ultrasonic and Coriolis flow meters, or a laser Doppler flow meter. The flow meter may determine a rate of fluid flow through a valve or other conduit and transmit a signal to a controller indicative of the determined flow rate.

Additionally, in some embodiments, a therapy unit otherwise like therapy unit 220 may further be configured to utilize wireless communication to communicate with a communications device. The wireless communication may allow for control of the therapy unit 220, monitoring the operation of the therapy unit 220, or both, via the wireless communication. In some embodiments, the therapy unit 220 may further include a communications transceiver. The communications transceiver may be configured to communicate via a suitable wireless communications protocol, for example, via Wi-Fi connection, a cellular connection, a Bluetooth® connection, a ZigBee®, with the communications device, such as a phone, a tablet, a pager, a personal computer, a server, or the like.

In various embodiments, the controller 224 may be a computing device or system, for example, a programmable logic controller or a data processing system. In some embodiments, the controller 224 may be a data processing system. The data processing system suitable for storing and/or executing program code may include at least one processor coupled directly or indirectly to memory elements through a system bus. The memory elements can include local memory employed during actual execution of the program code, bulk storage, and cache memories which provide temporary storage of at least some program code in order to reduce the number of times code is retrieved from bulk storage during execution.

In some embodiments, the controller 224 may be a programmable logic controller (PLC). The PLC may be a digital computer configured to receive one or more inputs and send one or more outputs in response to the one or more inputs. The PLC may include a non-volatile memory configured to store programs or operational instructions. In some embodiments, the PLC may be configured to receive discrete signals and continuous signals and produce discrete and continuous signals in response.

In some embodiments, the controller 224 may be configured to receive inputs from one or more other components, for example, from the negative-pressure source 222, the pressure sensor 226, the engagement sensor 228, and/or the user interface 230. Additionally, in some embodiments, the controller 224 may receive input, such as an electrical signal, from a source, such as through an electrical port. In some embodiments, the controller 224 may be configured to use various inputs to generate an output signal to another component, for example, a signal configured to operate that component. For example, a signal transmitted by the controller 224 to the negative-pressure source 222, which may be referred to herein as the controller 224 operating the negative-pressure source 222, may include signals effective to cause the negative-pressure source 222 to operate so to reduce the pressure within the canister 200 at a particular rate, to cause the negative-pressure source 222 to increase the rate at which the pressure within the canister 200 is reduced via the operation of the negative-pressure source 222, to cause the negative-pressure source 222 to decrease the rate at which the pressure within the canister 200 is reduced via the operation of the negative-pressure source 222, or to cause the negative-pressure source 222 to cease operation, for example, so as to control the pressure within the canister 200.

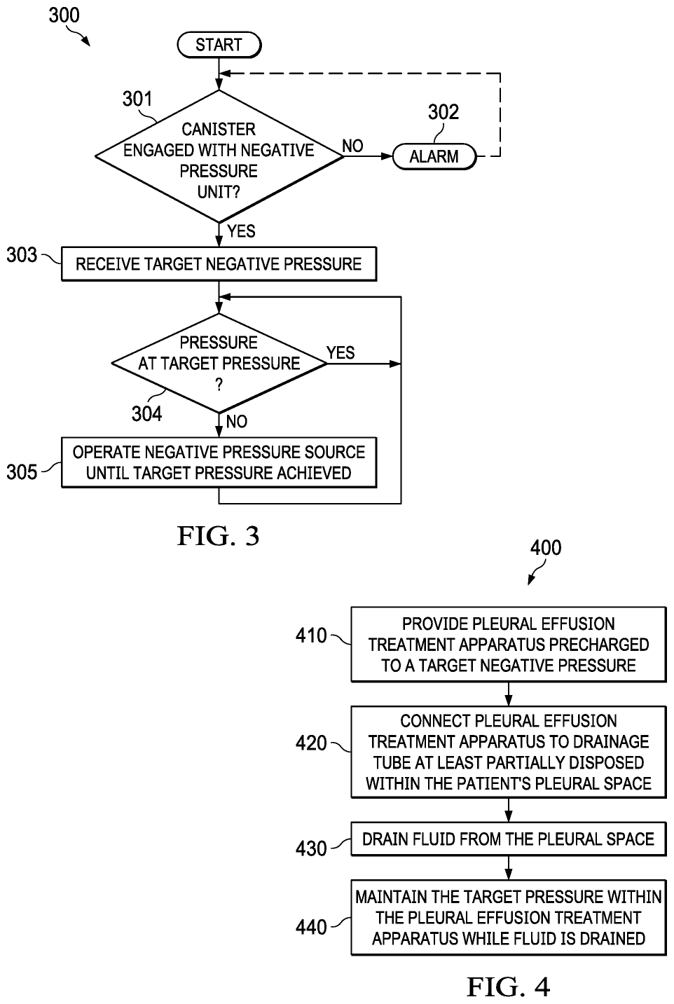

In some embodiments, the controller 224 is generally configured to cause the negative-pressure source 222 to be operated such that the internal space of the canister is reduced to and maintained within a desired negative-pressure range or beneath a target negative pressure. For example, referring to FIG. 3, a series of logical operations representing a process 300 that may be performed by the controller 224 is illustrated. In the embodiment of the FIG. 3, the process 300 performed by the controller 224 begins with the determination, in block 301, of whether or not the canister 200 is engaged with the therapy unit 220. If the controller 224 determines that the canister 200 is not engaged with the therapy unit 220, for example, if the controller 224 receives a signal from the engagement sensor 228 so-indicating, the controller 224 may provide, in block 302, an alarm, such as an indicator light, an icon on a touch screen, an audible alarm, a tactile or vibratory alarm, or a message transmitted to a remote unit. In some embodiments, the controller 224 will not allow the process 300 to progress further until the canister 200 is engaged with the therapy unit 220.

If the controller 224 determines that the canister 200 is engaged with the therapy unit 220, for example, if the controller 224 receives a signal from the engagement sensor 228 so-indicating, the controller 224 may receive, in block 303, one or more inputs from the external environment, for example, inputs related to a therapy regime. Particularly, in the embodiment of FIG. 3, the data may include a target negative pressure or, additionally or alternatively, a negative-pressure range, at which the pleural effusion treatment apparatus 120 is intended for use. In an alternative embodiment, the controller 224 may recall such data from a prior use or from a default series of data.

Referring again to FIG. 3, the process 300 progresses to the determination, in block 304, of whether or not the pressure of the internal space defined by the canister 200 is at the target pressure or within the defined negative-pressure range. For example, if the controller 224 receives a signal from the pressure sensor 226 indicating that the pressure within the canister 200 is greater than the target pressure, the controller 224 may operate the negative-pressure source 222, at block 305, so as to reduce the pressure within the canister 200 to the target pressure. Upon achieving the target pressure, the controller 224 may continue to monitor the pressure within the canister 200, for example, via the pressure sensor 226, to ensure that the pressure within the canister 200 remains substantially at the target pressure. In various embodiments, the controller 224 may continue to so-operate the negative-pressure source 222 for a predetermined duration or, indefinitely, such as until powered off or interrupted, for example, via an input from the user interface.

Additionally, in some embodiments, the controller 224 may be further configured to detect the presence of a leak from or within the system 100, such as from or within the canister 200 or another component in fluid communication therewith. For example, in operation, the only potential source of any substantial flow into the canister 200, such as an influx of air or liquid, should be the fluid flowing from the drainage fluid conductor. Therefore, in some embodiments, the controller 224 may be configured to recognize a series of conditions as indicative of a leak within the system 100, such as a pressure or fluid leak from or within the canister 200 or another component in fluid communication therewith. For example, the controller 224 may be configured to recognize operation of the negative-pressure source 222 that should theoretically exceed the internal volume of the canister 200 as indicating a leak. In such embodiments, the controller 224 may employ an algorithm effective to relate the operation of the negative-pressure source 222, the duration over which the negative-pressure source 222 is operated, and the internal volume of the canister 200. Thus, for example, such an algorithm may be effective to allow the controller 224 recognize that operation of the negative-pressure source 222 over a duration that would correspond to more fluid than might be held within the canister 200 and, thus, a leak. In some embodiments, when the controller 224 determines the presence of a leak, the controller may be further configured to provide an alarm, such as an indicator light, an icon on a touch screen, an audible alarm, a tactile or vibratory alarm, or a message transmitted remotely, for example, via the communication transceiver. Additionally or alternatively, when the controller 224 determines the presence of a leak, the controller 224 may be further configured to discontinue operation of the therapy unit 220, for example, to discontinue fluid communication with the drainage fluid conductor 110 and/or to shut-down the negative-pressure source 222.

Additionally, in some embodiments, the controller 224 may be further configured to determine the volume of fluid received into the canister, for example, from the patient. For example, in some embodiments, the controller 224 may employ such a determination, that is, the volume of fluid within the canister 200, to further determine if the canister 200 is full or nearly full or to monitor the total volume of fluid received from a patient over a given duration. In such embodiments, the controller 224 may employ an algorithm effective to determine the volume within the canister that is not occupied by fluid (which may be referred to as the “empty-space,” “void-space,” or “dead-space”). In some embodiments, the controller 224 may calculate the volume of the unoccupied space within the canister 200 by supplying a negative-pressure to the canister 200 and then venting the canister for a predetermined time interval, such as 1 second. By monitoring the resultant change in pressure, such as rise in pressure, during the venting interval, the controller 224 may determine unoccupied volume within the canister 200. Based upon the unoccupied volume within the canister 200, the volume within the canister that is occupied by fluid may be determined (i.e., as the difference between the total volume and the unoccupied volume). In some embodiments, when the controller 224 determines that the volume of fluid within the canister 200 is substantially close to the internal volume of the canister 200, the controller 224 may be further configured to provide an alarm, such as an indicator light, an icon on a touch screen, an audible alarm, a tactile or vibratory alarm, or a message transmitted remotely, for example, via the communication transceiver. Additionally or alternatively, when the controller 224 determines that the canister 200 is full, the controller 224 may be further configured to discontinue operation of the therapy unit 220, for example, to discontinue fluid communication with the drainage fluid conductor 110 or to shut-down the negative-pressure source 222. Additionally or alternatively, the controller 224 may be further configured to log or track the volume of fluid drained from a patient over a particular duration, for example, per treatment, per day, or per week. In some embodiments, the controller 224 may log or record the volume of fluid present within the canister 200 immediately prior to the canister 200 being drained.

Additionally or alternatively, in some embodiments, the canister 200, the therapy unit 220, or both may further include one or more shut-off valves, for example, generally configured to block flow upon the fluid within the canister reaching a predetermined level or volume. Examples of such a shut-off valve include, but are not limited to, a float valve comprising a buoyant ball retained within a trap and configured such that, when fluid within the canister reaches a predetermined volume or level, the buoyant ball blocks a flow path into the canister 200, a flow path out of the canister 200, or both. One or more shut-off valves, as disclosed herein, may be provided as a fail-safe, for example, to ensure that fluid does not overflow the canister 200 and to ensure that fluid is not suctioned into the negative-pressure source 222. In some embodiments, the suction port may comprise a filter, for example, an inline hydrophobic filter, for example, as a further fail-safe to ensure that any fluid does not reach that negative-pressure source 222.

### Intermediate Fluid Conductor

Referring again to FIG. 1, the intermediate fluid conductor 115 is representative of a fluid conductor generally configured to provide a route of fluid communication between the drainage fluid conductor 110 and the canister 200. As previously disclosed, in various embodiments, the intermediate fluid conductor 115 may be any suitably configured tube, pipe, hose, conduit, or other structure having one or more lumina adapted to convey a fluid between two ends thereof. In some embodiments, the intermediate fluid conductor 115 may include suitably-configured connectors at the terminal ends thereof.

Additionally, in some embodiments, the intermediate fluid conductor 115 may include a valve. For example, in some embodiments, one or more of the connectors at the terminal ends of the intermediate fluid conductor 115 may include a valve, for example, adapted so as to disallow fluid communication in at least one direction, such as fluid flow in the direction of the canister 200, when the drainage fluid conductor 110 is not coupled with a connector of the intermediate fluid conductor 115 and to allow fluid communication when drainage fluid conductor 110 is coupled with a connector of the intermediate fluid conductor 115. In some embodiments, the valve may be a pinch valve. A pinch valve may be a portion of a fluid conductor having a clamping device positioned there-about so as to selectively compress the fluid conductor so as to block the passage of fluid through that fluid conductor. In some embodiments, the portion of a fluid conductor having the clamp valve may be formed of a suitable elastomeric composition, for example, silicone.

### Drainage Fluid Conductor

The drainage fluid conductor 110 is also representative of a fluid conductor that may be generally configured to provide fluid communication with the pleural space of a patient. For example, at least a portion of drainage fluid conductor 110 may be configured for implantation within a patient, for example, within the pleural space of the patient. In some embodiments, the drainage fluid conductor 110 may comprise or consist essentially of a manifold 111, for example, generally comprising an elongated, flattened or ribbon-like conduit having a plurality of apertures. For example, such a plurality of apertures along the implantable, manifolded portion of the drainage fluid conductor 110 may allow fluid to flow into a flow-space of the drainage fluid conductor 110. Suitable examples of a drainage fluid conductor include a chest tube, a chest drain, a thoracic catheter, or an intercostal tube.

### Pleural Effusion Treatment Method 1 (for System 1)

Also disclosed herein are embodiments of methods of treating a plural effusion, for example, employing a system such as the system 100 disclosed herein. Referring to FIG. 4, an embodiment of a pleural effusion treatment method 400 is illustrated. In the embodiment of FIG. 4, the pleural effusion treatment method 400 generally includes the steps of providing a pleural effusion treatment apparatus precharged to a target negative pressure 410, connecting the pleural effusion treatment apparatus to a drainage fluid conductor having a portion thereof in fluid communication with the pleural space of a patient 420, draining fluid from the pleural space of the patient 430, and maintaining the target negative pressure while fluid is drained 440. Additionally, in some embodiments, the pleural effusion treatment method 400 may further comprise the step of draining the canister, for example, such that fluid may continue to be drained from the pleural space.

In some embodiments, in operation, a pleural effusion treatment apparatus like the pleural effusion treatment apparatus 120 disclosed with respect to FIG. 1 may be precharged to a target negative pressure. For example, a user, such as a physician or other care provider may input the target negative pressure and/or a target negative pressure range via the user interface 230 and the therapy unit 220 may operate to charge the canister 200 to the target negative pressure and/or target negative-pressure range. Once the target negative pressure and/or target negative-pressure range is achieved, the pleural effusion treatment apparatus 120 may be stored until it is needed for use. During storage, the therapy unit 220 may maintain the canister 200 at the target negative pressure and/or within target negative-pressure range, for example, such that the pleural effusion treatment apparatus 120 will be precharged at the negative pressure when needed.

In some embodiments, when a patient experiencing a pleural effusion is in need of therapy, the pleural effusion treatment apparatus 120 may be provided for use. In some embodiments, the therapy may commence with the implantation of the drainage fluid conductor 110, for example, by a physician. For example, the physician may make one or more incisions, for example, between the midaxillary and anterior axillary lines, so as to allow access to the pleural space and may implant at least a portion of the drainage fluid conductor, such as the free terminal end including the manifolded portion of the drainage fluid conductor, within the pleural space of a patient in need of therapy.

In some embodiments, with the drainage fluid conductor 110 implanted within pleural space of the patient, the drainage fluid conductor 110 may be fluidly connected to the pleural effusion treatment apparatus 120, for example, such that there is a route of fluid communication via the drainage fluid conductor 110 between the canister 200 and the pleural space. For example, in various embodiments, a physician, caregiver, or the patient may connect the drainage fluid conductor 110 to the canister 200, for example, either directly or via one or more intermediate fluid conductors 115. In some embodiments, with the drainage fluid conductor 110 connected to the canister 200, the physician, caregiver, or patient may enable fluid communication between the pleural space and the canister 200, for example, such that the negative pressure from the canister 200 is applied to the pleural space of the patient. In various embodiments, enabling fluid communication between the canister 200 and the pleural space may be achieved manually or automatically and, for example, may include opening a valve or clamp, entering a command via the user interface 230, or responding to a prompt via a user interface 230 or remote control, such that the controller causes a valve or other flow control device to open.

In some embodiments, as the therapy progress, fluid may be drained from the pleural space of the patient and conveyed into the canister 200. As the fluid drains, the inflow of fluid from the pleural space into the canister 200 may result in an increase in the pressure within the canister 200. As previously disclosed herein, the controller 224 may be configured to cause the negative-pressure source 222 to maintain the negative pressure within canister 200 while fluid is drained from pleural space. For example, as the negative pressure decreases, the negative-pressure source 222 may operate so as to maintain the canister 200 at substantially the target negative pressure and/or within target negative-pressure range. The negative-pressure source 222 may continue to operate, for example, until the canister 200 becomes filled or until a problematic condition is detected by the controller 224.

In some embodiments, for example when the canister 200 has filled or when the therapy has concluded, such as after a prescribed duration, the pleural effusion treatment apparatus 120 may be disconnected and the canister 200 may be drained and cleaned, for example, sanitized. For example, draining and cleaning the canister may allow the canister to be reused, for example, if the therapy is to continue or, alternatively, in a later therapy.

In some embodiments, as the therapy progress, the controller 224 may detect the presence of a leak, such as a fluid or pressure leak, for example, a leak from or within the canister 200 or another component in fluid communication therewith. As previously disclosed, in some embodiments, when the controller 224 determines the presence of a leak, the controller 224 may cause an alarm to be issued. For example, the controller 224 may issue an indicator light, an icon on a touch screen, an audible alarm, a tactile or vibratory alarm, trigger a message transmitted remotely, for example, via the communication transceiver. Additionally or alternatively, the controller 224 may cause operation of the negative-pressure source 222 to be discontinued.

In some embodiments, as the therapy progress, the fluid drained from the pleural space of the patient and conveyed into the canister 200 may fill the canister 200, for example, to an extent that the canister 200 is full or substantially full. In some embodiments, when the controller 224 determines that the canister 200 is full, the controller 224 may cause an alarm to be issued or, additionally or alternatively, the controller may cause operation of the therapy unit to be discontinued.

### System 2

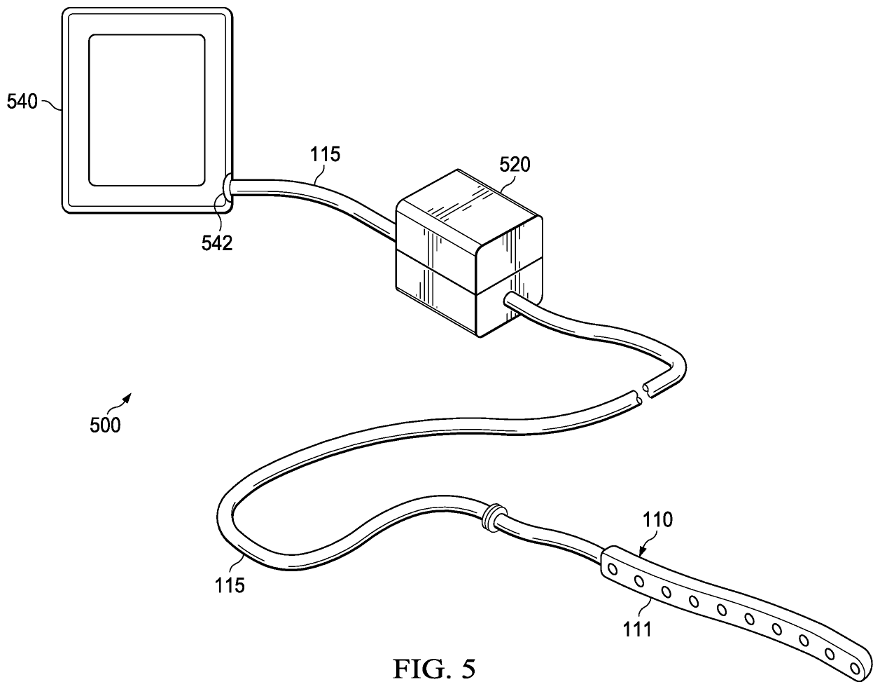

In some embodiments, a system may comprise a fluid conductor, such as a tube, configured to provide fluid communication with a pleural space of a patient, a pump head, a power unit detachably coupled to the pump head so as to impart rotational power to the pump head when coupled, and a collection vessel, the pump head being positioned along a route of fluid communication between the fluid conductor and the collection vessel. For example, FIG. 5 illustrates a second embodiment of a system 500 that can provide treatment or management of a pleural effusion. In the embodiment of FIG. 5, the system 500 generally comprises a drainage fluid conductor 110, a pump unit 520, and a collection vessel 540. Also in the embodiment of FIG. 5, the system 500 may include one or more intermediate fluid conductors 115 adapted to provide fluid communication between the drainage fluid conductor 110 and the collection vessel 540 via the pump unit 520. As similarly noted with respect to the embodiments of FIG. 1, the intermediate fluid conductor 115 may be omitted or may be present in any suitable number. The drainage fluid conductor 110 and intermediate fluid conductors 115 may be suitably configured, for example, as similarly disclosed with respect to the system of FIG. 1.

### Collection Vessel

In some embodiments, the collection vessel 540 may be generally configured to receive and retain a fluid, such as fluid from the pleural space. The collection vessel 540 may receive the fluid from the pump unit 520, when the pump unit 520 is operated. In some embodiments, the collection vessel 540 may be configured define a variable internal volume. For example, in the embodiment of FIG. 5, the collection vessel 540 comprises a flexible bag or pouch that is configured to be expandable, for example, such that in an expanded state or configuration, the bag or pouch will retain a maximum volume.

An expandable collection vessel, like the collection vessel 540 of FIG. 5, may have any suitable, maximum internal volume. For example, in various embodiments, the collection vessel 540 may have a maximum internal volume of from about 0.5 L to about 2.5 L. In various embodiments where collection vessel 540 is expandable, the collection vessel 540 may be formed from any suitable material or assemblage of materials. For example, the collection vessel, which may be in the form of a bag or pouch, may comprise a suitable film material, such as a plastic, resin-based film, that may be formed into the sealed bag or pouch. For example, in such some embodiments, the bag or pouch may be formed by joining two or more sheets, for example, via a weld, such as between thermoplastic materials. Examples of materials that may be used to form such the collection vessel 540 in embodiments where the collection vessel 540 is expandable, may include, but are not limited to, films such as low-density linear polyethylene (LLDPE), low-density polyethylene (LDPE), high-density polyethylene (HDPE), polypropylene (PP), polyvinyl chloride (PVC), ethylene vinyl acetate (EVA), polyester, polyurethane (PU or PUR), or combinations thereof.

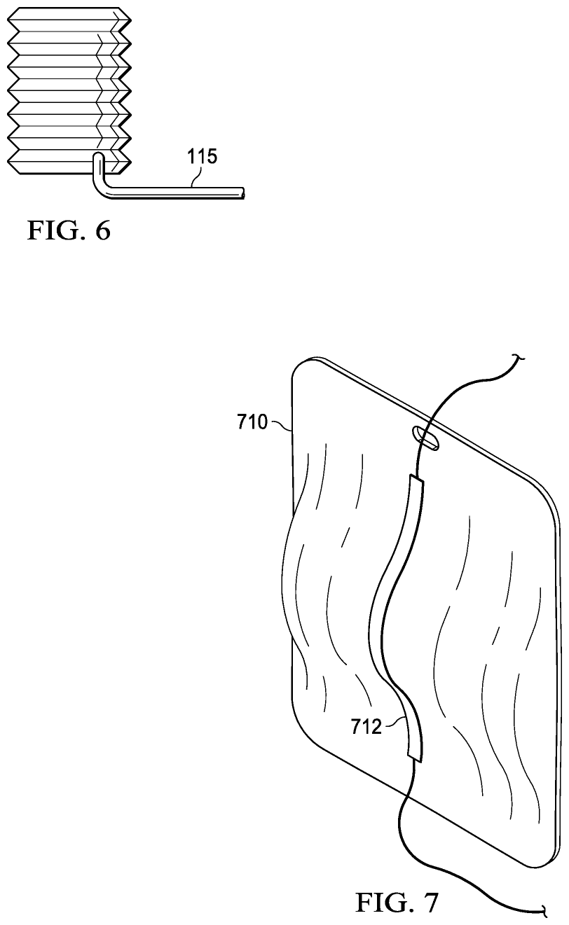

In an alternative embodiment, a collection vessel may comprise a bellows-container, for example, a bottle having one or more collapsible, accordion-like, circumferential walls enabling the bottle to be axially expanded or contracted. Referring to FIG. 6, an embodiment of such a bellows-container is illustrated.

In an alternative embodiment, a collection vessel may comprise a rigid container, for example, having a fixed internal volume, for example, an internal volume of from about 0.5 L to about 2.5 L.

In some embodiments, the collection vessel 540 or another suitably-configured collection vessel may be transparent or translucent, for example, to enable viewing of the fluid contained therein. Additionally or alternatively, in some embodiments, the collection vessel 540 may include one or more sight-windows to similarly enable viewing the fluid. Also, in various embodiments, the collection vessel may include various markings, for example, indicating the volume of fluid therein.

In some embodiments, the collection vessel 540 may comprise one or more connection ports, for example, generally configured to provide a fluid connection so as to receive fluid into the internal volume or to allow fluid retained within the internal volume of the collection vessel 540 to be drained therefrom. In some embodiments, the connection ports may be suitably located with respect to the collection vessel 540. For example, in the embodiment of FIG. 5, the collection vessel 540 comprises a connection port 542 within a sidewall toward the base of the collection vessel 540. Alternatively, a connection port may be provided at the top of a collection vessel, at the bottom of the collect vessel, or within a sidewall toward the top of the collection vessel. Additionally, in some embodiments, the connection port 542 may include a valve, for example, adapted so as to disallow fluid communication in at least one direction when a tubing is not coupled with the connection port 542 and to allow fluid communication when a tubing is coupled with the connection port 542. In such embodiments, fluid flow into the collection vessel 540 via the connection port 542 may be precluded via the valve when a tubing such as the drainage fluid conductor 110 or the intermediate fluid conductor 115 is not connected to the connection port 542. For example, in some embodiments the connection port 542 may be configured as a “duck-billed valve, a float valve, or a flap valve.

Additionally, in some embodiments, a collection vessel like collection vessel 540 may be configured for reuse. For example, in some embodiments, the collection vessel 540 may be configured such that a fluid retained therein may be drained. For example, in some embodiments, the connection port 542 may be configured to allow a fluid to be drained therefrom, for example, via a drain-tube or “tail tube.” Additionally or alternatively, in some embodiments, a collection vessel may include a drain port. In such embodiments, the drain port may be located toward the bottom of the collection vessel, for example, and may be adapted to allow a fluid contained within the internal volume of the collection vessel to be drained therefrom. The drain port may be fitted with a removable plug or may include a valve, such as a gate valve or petcock valve, that may be opened to allow fluid to be drained from the internal volume of the collection vessel.

Alternatively, in other embodiments, a collection vessel may be configured for a limited number of uses, for example, a single-use, double-use, 5-use, 10-use, 15-use, or 20-use collection vessel. In such embodiments, the collection vessel may be configured such that at least a portion of a fluid received and retained therein may be evaporated therefrom. For example, the collection vessel may comprise one or more moisture-vapor permeable surfaces having a high moisture-vapor transmission rate (MVTR). For example, the MVTR may be at least 300 g/m² per twenty-four hours, in some embodiments. In some embodiments, the moisture-vapor permeable surface may be a polymeric film, such as a polyurethane film, that is permeable to water vapor but impermeable to liquid. Such films may typically have a thickness in the range of 25-50 microns. Additionally or alternatively, the collection vessel may comprise one or more lumens generally configured to allow air-flow, for example, to encourage evaporation from the collection vessel. In such embodiments, the air flow may be from naturally circulating air or from a forced-air source, such as a fan. Additionally, the collection vessel may include a distribution layer, for example, to increase the surface area of the pouch with which the fluid is in contact.

In some embodiments, the collection vessel 540 may comprise a fluid manifold generally configured to improve fluid communication between the internal volume of the collection vessel and a port, for example, the connection port 542. For example, the fluid manifold may comprise one or more layers disposed within the internal volume of the collection vessel. For example, such a fluid manifold may be formed from a woven material such as Libeltex TDL2, a non-woven material, one or more polymeric sheets, cast- or injection-molded polymer resin, or a foam material disposed within the internal volume of the collection vessel.

Also, in some embodiments, the collection vessel 540 may include a hydrophobic coating or an anti-fowling coating, for example, to enable multiple uses of the collection vessel 540. In such embodiments, the coating may be applied to the surfaces intended for contact with a fluid from the pleural space, for example, the interior or flow-spaces.

In some embodiments, the collection vessel 540 may be configured to provide feedback, such as to output a signal, for example, to the pump unit 520, to a controller, or to a user interface, as will be disclosed herein. For example, the collection vessel 540 may be configured to provide a signal indicative of fill level. In such embodiments, the collection vessel may comprise, for example, a strain gauge or Hall Effect sensor configured to sense fill level and to output a signal indicative of the same. For example, FIG. 7 illustrates an example embodiment of a collection vessel 710 comprising a strain gauge 712. In the embodiment of FIG. 7, the strain gauge 712 is attached to the side of the collection vessel 710 such that, as the collection vessel 710 is filled with a fluid, such as fluid from the pleural space, the collection vessel 710 undergoes an increasing deflection, causing the electrical resistance through the strain gauge 712 to vary and, thus outputting a signal corresponding to the deflection of the collection vessel 710, and thus the fill level.

In some embodiments, for example, in embodiments where the collection vessel comprises a rigid container, the collection vessel may further comprise a vent or filter, for example, configured to allow pressure to dissipate from the collection vessel as fluid is conveyed therein, such as fluid from the pleural space. In some embodiments, the vent may comprise a filter, for example, a hydrophobic filter, for example, generally configured to allow the passage of air but not liquids. The hydrophobic filter may be, for example, a porous, sintered polymer cylinder sized to fit the dimensions of the vent so as to substantially preclude liquid from passing through the vent.

### Pump Unit

In some embodiments, the pump unit 520 may generally include a pump head generally configured to, when operated, cause a fluid to be conveyed therethrough and a power unit generally configured to supply power to the pump head. For example, in the embodiment of FIG. 5, the pump unit 520 generally includes a pump head 525 and a power unit 530. In the embodiment of FIG. 5, the pump unit 520 is positioned in-line, for example, by way of the intermediate fluid conductors 115, between the drainage fluid conductor 110 and the collection vessel 540 such that, when operated, the pump unit 520 develops negative pressure that is applied to the drainage fluid conductor 110. The pump unit 520 is positioned in-line such that, for example, fluid be drawn drawn into the drainage fluid conductor 110 and conveyed to the collection vessel 540. For example, the fluid may be conveyed through the intermediate fluid conductors 115 and the pump unit 520, more particularly, through the pump head 525,

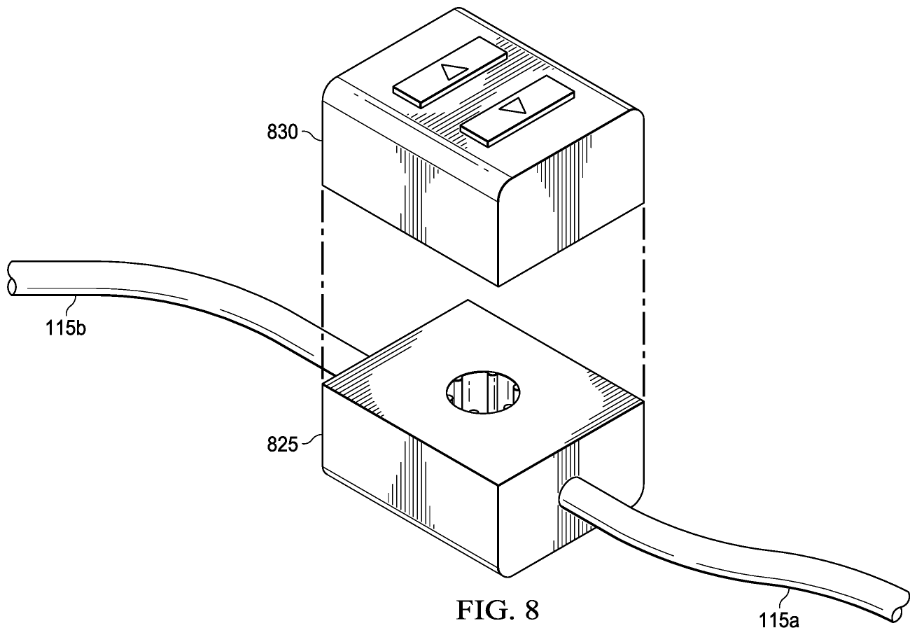

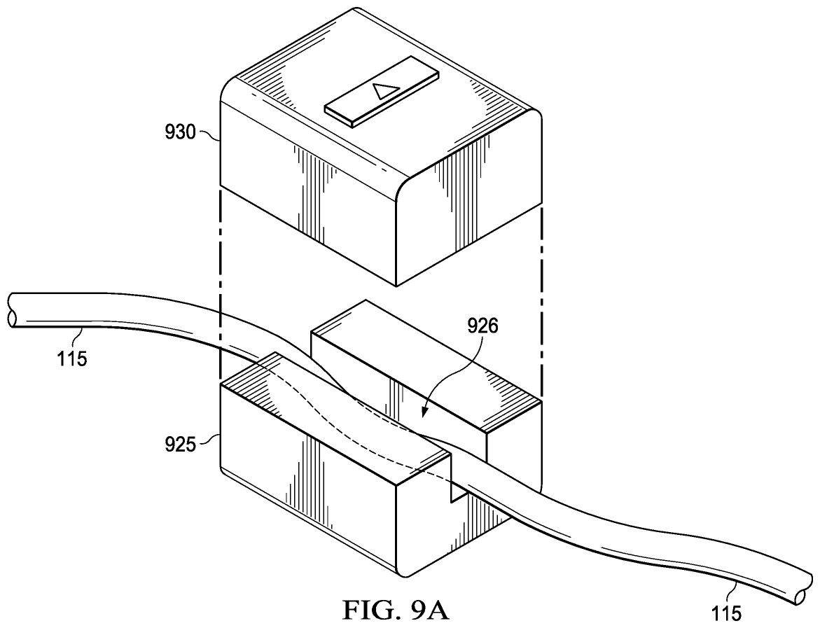

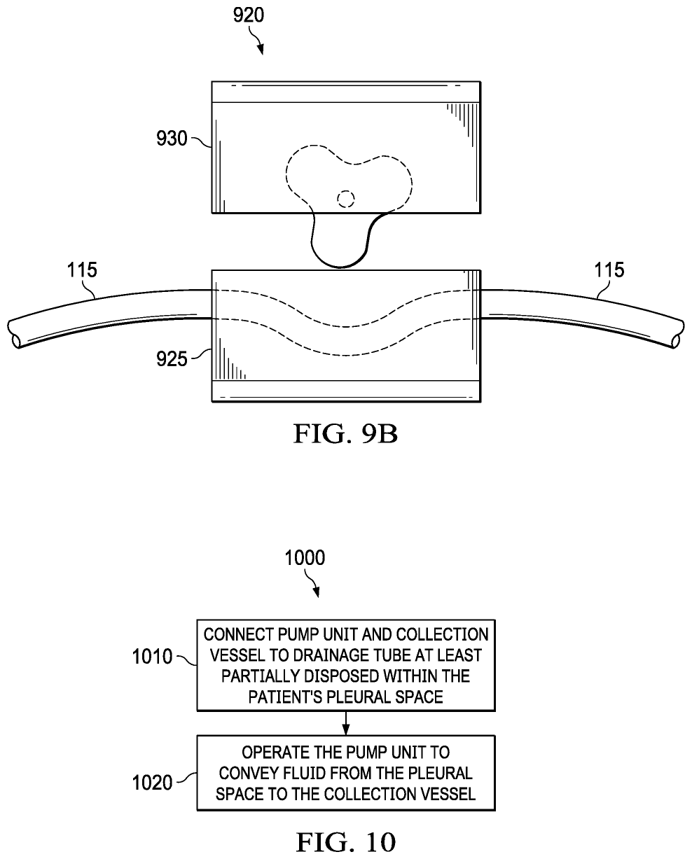

Generally, the power unit 530 may be coupled, for example, releasably coupled, to the pump head 525 such that the power unit 530 supplies power, for example, rotational power, to the pump head 525. For example, the power unit 530 may apply power to the pump head so as to cause the pump head 525 to operate. FIGS. 8, 9A and 9B illustrate example embodiments of a pump unit like pump unit 520.

### Pump Head

The pump head 525 may be generally configured, when operated, to draw a fluid therethrough. In some embodiments, the pump head 525 may be configured for bi-directional fluid movement, for example, to selectively convey fluid in either a first direction or a second, opposite direction, with respect to the pump head. For example, in some embodiments, the pump head may be configured such that, rotational motion applied to the pump head in a first rotational direction causes the pump head to operate such that fluid is conveyed in a first direction and such that rotational motion applied to the pump head in a second rotational direction causes to the pump head to operate such that fluid is conveyed in a second, opposite direction. Alternatively, in another embodiment the pump head may be configured to receive rotational motion in only a single direction and, further, to selectively operate such that fluid is conveyed in a first direction or such that fluid is conveyed in a second, opposite direction. For example, in such embodiments the pump head may comprise a “shift” lever, for example, to alter the operation of the pump head between “forward” fluid movement and “reverse” fluid movement.

Alternatively, in other embodiments, the pump head 525 may be configured for uni-directional fluid movement (that is, to convey fluid in only a single direction, with respect to the pump head). While a pump head may be configured for only uni-directional fluid movement, such a uni-directional pump head may nonetheless be employed to convey fluid in two, opposite direction, for example, by altering the arrangement of the pump head 525, such as the various fluid connections to or from the pump head. For example, in the contact of FIG. 5, in embodiments where the pump head 525 is configured to operate uni-directionally, the pump head 525 might be operated so as to convey fluid into the collection vessel 540 or out of the collection vessel dependent upon the fluid connection to the pump head 525.

In various embodiments, the pump unit 520 may be configured as any suitable pump type or configuration, examples of which include, but are not limited to, a peristaltic pump, a rotary pump, an impeller pump, a lobe pump, and a screw pump or twin screw pump. In some embodiments, the pump head may be configured for connection to a fluid conductor, such as the drainage fluid conductor 110 or an intermediate fluid conductor 115, such that fluid is drawn directly into the pump head, for example, into a pump chamber. For example, in the embodiment of FIG. 8, the pump head 825 is attached to and in an fluid communication with each of a first intermediate fluid conductor 115a and a second intermediate fluid conductor 115b such that fluid from either the first or second intermediate fluid conductor, 115a or 115b, respectively, may be conveyed into a pump chamber within the pump head 825 and from to pump chamber to the other of the first or second intermediate fluid conductor, 115a or 115b, respectively. In such embodiments, the pump head 825 may comprise connection ports suitable for making a fluid connection to the intermediate fluid conductor 115 or the drainage fluid conductor 110.

Alternatively, in some embodiments, the pump head may be configured to receive a fluid conductor such that fluid from the fluid conductor does not directly contact the pump head or any component thereof. For example, referring to FIGS. 9A and 9B, an embodiment of a pump unit 920 configured as a peristaltic pump is shown. In the embodiment of FIGS. 9A and 9B, a fluid conductor, for example, the intermediate fluid conductor 115, is received into a trough 926 in the pump head 925. The trough generally comprises a longitudinal, centrally-located, semi-circular valley. The pump head 925 may be joined with the power unit 930 which comprises a rotor having a number of lobes that, when the rotor is rotated, cause fluid to be conveyed through the tubing within the pump head 925 without the fluid directly contacting the pump head.

In some embodiments, the pump head may be configured to be reusable. For example, in some embodiments, the pump head in configured so as to be easily disassembled, for example, such that any components in contact with fluid may be cleaned or replaced.

### Power Unit

A power unit like power unit 530, power unit 830, or power unit 930 is generally configured to supply power, particularly, rotational power, to the pump head, for example, pump head 525, 825, or 925, respectively. In various embodiments, the power unit may use any suitable source of power to supply the rotational power. For example, the power unit 530 may derive power from a wall outlet, battery, or any other suitable source.

Also, the power unit 530 may be configured to convey the rotational power to the pump head. For example, in the embodiment of FIG. 8, the power unit 830 is configured to convey rotational power to the pump head 825 via a rotational interface, for example, a Hirth joint, a keyed or splined-shaft coupling, a box or sleeve coupling, a disc coupling, a geared coupling, a magnetic coupling, or the like. Alternatively, in the embodiment of FIG. 9, the power unit 930 is configured to convey rotational power to the pump head 925 via a peristaltic pump configuration.

The power unit 530 may also be generally configured to be controlled or operated by a user. For example, the power unit 530 may comprise any suitable user interface, as previously disclosed herein. In some embodiments, such as in the embodiment of FIG. 8, the power unit 830 comprises a first button and a second button, for example, push-and-hold buttons or toggle buttons. The first button may be configured such that, when actuated, the power unit 830 is caused to operate in a first rotational direction, for example, such that the pump head 825 operates so as to convey fluid from the drainage fluid conductor 110 to the collection vessel 540. The second button may be configured such that, when actuated, the power unit 830 is caused to operate in a second rotational direction, for example, such that the pump head 825 operates so as to convey fluid out of the collection vessel. In alternative embodiments, such as in the embodiment of FIGS. 9A and 9B, the power unit 930 may comprise only a single button that, when actuated causes the power unit 930 to operate in only a first direction. In various embodiments, the power unit may also be configured to allow for variable operational speeds.

In an alternative embodiment, the power unit may be configured for manual operation, for example, such that a user may supply the power, for example, via an input device. For example, the user may impart rotational motion to the pump head. In such embodiments, the power unit may comprise a Yankee screw, a crank, or a lever, for example.

In an additional embodiment, a power unit like power unit 530, power unit 830, or power unit 930 may further comprise a controller. In such embodiments, the controller may be configured as previously disclosed herein, as suitable. In some embodiments, the controller may be configured to detect the presence of a blockage. For example, in such embodiments, the controller may be configured to log a standard or “benchmark” current draw associated with operation of the pump unit, such as the current draw under normal operating conditions. The controller may also be configured to recognize deviations from the normal current draw. An increase in the current draw associated with operation of the pump unit may be indicative of one or more potentially problematic conditions, for instance, a blockage within the drainage fluid conductor or intermediate fluid conductor, the collection vessel being full, or (if the pump is being used to drain fluid from the collection vessel) the collection vessel being empty. In some embodiments, when the controller recognize an increase in current draw, the controller may be further configured to provide an alarm, such as an indicator light, an icon on a touch screen, an audible alarm, a tactile or vibratory alarm, or a message transmitted remotely, for example, via the communication transceiver. Additionally or alternatively, when the controller recognizes an increase in current draw, the controller may be further configured to discontinue operation of the pump unit.

### Pleural Effusion Treatment Method 2 (for System 2)

Also disclosed herein are additional embodiments of methods of treating a plural effusion, for example, employing a system such as the system 500 disclosed herein. Referring to FIG. 10, an embodiment of a pleural effusion treatment method 1000 is illustrated. In the embodiment of FIG. 10, the pleural effusion treatment method 1000 generally includes the steps of connecting a pump unit and a collection vessel to a drainage fluid conductor having a portion thereof in fluid communication with the pleural space of a patient 1010 and operating the pump unit to convey fluid from the pleural space to the collection vessel 1020.

In some embodiments, in operation, when a patient experiencing a pleural effusion is in need of therapy, therapy may commence with the implantation of a drainage fluid conductor like the drainage fluid conductor 110 of FIG. 5, for example, by a physician. For example, the physician may implant at least a portion of the drainage fluid conductor 110, such as the free terminal end, including the manifolded portion of the drainage fluid conductor, within the pleural space of a patient in need of therapy.

In some embodiments, with the drainage fluid conductor 110 implanted within pleural space of the patient, the pump unit 520 and collection vessel 540 may be connected to the drainage fluid conductor 110. For example, in various embodiments, a physician, caregiver, or the patient may connect the drainage fluid conductor 110 to the collection vessel 540, for example, optionally, via one or more intermediate fluid conductors 115, for example, such that there is a route of fluid communication between drainage fluid conductor 110 and the collection vessel 540. Also, the physician, caregiver, or patient may connect the pump unit 520 such that the pump unit 520 is positioned between the drainage fluid conductor 110 and the collection vessel 540. In some embodiments, the pump unit 520 may be connected such that the pump head 525 is in fluid communication with the drainage fluid conductor 110 and collection vessel 540, for example, as disclosed with respect to FIG. 8. Alternatively, in some embodiments, the pump unit 520 may be connected such that the pump head 525 is not in fluid communication with the drainage fluid conductor 110 and collection vessel 540, for example, as disclosed with respect to FIGS. 9A and 9B. Also, in some embodiments, for example, where the collection vessel 540 comprises a bellows-container, the pump unit 520 may be used to “prime” the collection vessel, for example, such that the collection vessel 540 may be at a negative pressure, prior to fluidly connecting the collection vessel 540 to the drainage fluid conductor 110.

In some embodiments, with the pump unit 520 and collection vessel 540 fluidly connected to the drainage fluid conductor 110, the pump unit 520 may be operated so as to convey fluid from the pleural space to the collection vessel 540. For example, and not intending to be bound by theory, operation of the pump unit 520 may be effective to cause a negative pressure to be applied to the pleural space, for example, so as to withdraw fluid from the pleural space and to convey the fluid from the pleural space to the collection vessel 540.

In some embodiments, for example when the collection vessel 540 has filled or when the therapy has concluded, such as after a prescribed duration, the collection vessel 540 may be disconnected and may be drained and cleaned, for example, sanitized. For example, draining and cleaning the canister may allow the collection vessel 540 to be reused, for example, if the therapy is to continue or, alternatively, in a later therapy. Alternatively, in some embodiments, the collection vessel 540 may be disposable, and may be replaced in futures provisions of pleural effusion treatment.

Additionally, in some embodiments, the pump unit 520 may be disassembled and the pump head 525 cleaned, for example, such that the pump head 525 may be reused. Alternatively, in some embodiments, the pump head 525 may be disposable, and may be replaced in futures provisions of pleural effusion treatment.

### ADVANTAGES

In various embodiments, a system like system 100 or system 500, or components thereof, may be advantageously employed in the provision of therapy to a patient experiencing a pleural effusion, that is, pleural effusion treatment. Particularly, while conventional charged canisters are intended only for a single-use and cannot be recharged and reused, the disclosed systems and apparatuses like systems 100 and/or 500 and the associated apparatuses are reusable. Thus, conventionally, a patient requires a new charged canister for each treatment. This presents a significant cost to the healthcare system and, moreover, is a major inconvenience to patients and healthcare providers, who are tasked with ensuring the availability of sufficient numbers of charged canister and disposing of used canisters. Thus, the significant costs and inconveniences associated with providing and disposing of such conventional canisters can be alleviated significantly through use of the disclosed systems like systems 100 and/or 500 and the associated apparatuses, based upon their reusability.

Moreover, the quality of therapy provided by the disclosed systems and apparatuses like systems 100 and/or 500 and the associated apparatuses is significantly improved with respect to conventional systems and apparatuses. Particularly, with conventional canisters, the in-flow of fluid into the canister results in an increase in the pressure therein, meaning that the negative pressure experienced by the patient decreases as the treatment progresses and that the negative pressure available to draw excess fluid from the pleural space is least toward the end of a given treatment. However, later in a given treatment is when negative pressure applied to the pleural space would be most beneficial (because this is when there is the relatively less excess fluid). Advantageously, with the disclosed systems 100 and/or 500 and the associated apparatuses, the target negative pressure and/or negative-pressure range is maintained throughout the course of the therapy.

While shown in a few illustrative embodiments, a person having ordinary skill in the art will recognize that the systems, apparatuses, and methods described herein are susceptible to various changes and modifications. Moreover, descriptions of various alternatives using terms such as “or” do not require mutual exclusivity unless clearly required by the context, and the indefinite articles “a” or “an” do not limit the subject to a single instance unless clearly required by the context. Components may be also be combined or eliminated in various configurations for purposes of sale, manufacture, assembly, or use. For example, in the system 100 of FIG. 1, a therapy unit and canister may be separated from each other components for manufacture or sale. Likewise, in the system 500 of FIG. 5, a collection vessel and pump unit and/or the components of the pump unit may be separated from each other components for manufacture or sale. In other example configurations, a controller as disclosed herein may also be manufactured, configured, assembled, or sold independently of other components.

The appended claims set forth novel and inventive aspects of the subject matter described above, but the claims may also encompass additional subject matter not specifically recited in detail. For example, certain features, elements, or aspects may be omitted from the claims if not necessary to distinguish the novel and inventive features from what is already known to a person having ordinary skill in the art. Features, elements, and aspects described herein may also be combined or replaced by alternative features serving the same, equivalent, or similar purpose without departing from the scope of the invention defined by the appended claims.

1. A system for the treatment of pleural effusion, the system comprising:
a fluid conductor configured to provide fluid communication with a pleural space of a patient;
a fluid container fluidly coupled to the fluid conductor; and
a negative-pressure source in fluid communication with the fluid container, the negative-pressure source being configured to precharge the fluid container to a negative-pressure range and to maintain the negative-pressure range within the fluid container while the fluid container and the fluid conductor are in fluid communication.

2. (canceled)

3. The system of claim 1, wherein the negative-pressure source comprises a pump and a controller, wherein the controller is configured to cause the pump to be operated such that the fluid container is maintained within the negative-pressure range, wherein the negative-pressure range is from about −50 mmHg to about −100 mmHg.

4. (canceled)

5. The system of claim 3, wherein the controller is configured to detect a leak from the fluid container or a component in fluid communication therewith.

6. The system of claim 3, wherein the controller is configured to determine a volume of a fluid received into the fluid container.

7. The system of claim 3, further comprising a valve configured to control fluid communication between the fluid container and the fluid conductor, wherein the controller is configured to send a signal effective to actuate the valve.

8.-11. (canceled)

12. The system of claim 1, wherein the fluid conductor comprises a manifold configured for placement within the pleural space of the patient.

13. (canceled)

14. (canceled)

15. A system for the treatment of pleural effusion, the system comprising:
a fluid conductor configured to provide fluid communication with a pleural space of a patient;
a pump head;
a power unit detachably coupled to the pump head so as to impart rotational power to the pump head when coupled; and
a collection vessel, the pump head being positioned along a route of fluid communication between the fluid conductor and the collection vessel.

16.-18. (canceled)

19. The system of claim 15, wherein the collection vessel comprises a first port in fluid communication with a first manifold within an internal volume of the collection vessel, wherein the collection vessel further comprises a second port in fluid communication with a second manifold within the internal volume of the collection vessel.

20. (canceled)

21. The system of claim 15, wherein the collection vessel is configured so as to allow at least a portion of a fluid contained within the collection vessel to be evaporated therefrom.

22. The system of claim 21, wherein the collection vessel comprises a semi-permeable membrane.

23. (canceled)

24. The system of claim 15, wherein the power unit is configured to impart rotational power to the pump head, when so-actuated, in a first direction of rotation and, when so-actuated, in a second direction of rotation.

25. The system of claim 15, wherein the power unit is configured to impart rotational power to the pump head, when actuated, in only a first direction of rotation.

26. The system of claim 25, wherein:
in a first configuration, the pump head is configured such that, upon receiving the rotational power in the first direction of rotation, fluid is communicated into the collection vessel, and
in a second configuration, the pump head is configured such that, upon receiving the rotational power in the first direction of rotation, the fluid is communicated out of the collection vessel.

27. The system of claim 15, wherein the pump head is configured for connection to the fluid conductor such that a chamber of the pump head is in fluid communication with a flowpath of the fluid conductor.

28. The system of claim 26, wherein the pump head is configured to receive at least a portion of the fluid conductor, and wherein the pump head is configured as a peristaltic pump.

29. (canceled)

30. The system of claim 15, wherein the pump head is in signal communication with a controller, wherein the controller is configured to detect a blockage within the fluid conductor or a component in fluid communication therewith.

31. (canceled)

32. The system of claim 30, wherein the controller is configured to determine a volume of a fluid received into the collection vessel.

33. The system of claim 15, wherein the fluid conductor comprises a manifold.

34. The system of claim 15, wherein the fluid conductor comprises an intercostal tube.

35. (canceled)

36. A method for treating pleural effusion, comprising:
providing a pleural effusion apparatus comprising:
a fluid conductor configured to provide fluid communication with a pleural space,
a fluid container fluidly coupled to the fluid conductor, and
a negative-pressure source in fluid communication with the fluid container;
connecting the pleural effusion apparatus to a drainage tube;
draining fluid from the pleural space; and
maintaining a target pressure range within the pleural effusion treatment apparatus while fluid is drained.
